(19) United States
(12) Patent Application Publication
(10) Pub. No.: US 2016/0250133 A1
(43) Pub. Date: Sep. 1, 2016

(54) HAIR TREATMENT AGENT COMPRISING POLYALKOXY ALKYL AMINO-SUBSTITUTED SILOXANES

(71) Applicant: Henkel AG & Co. KGaA, Duesseldorf (DE)

(72) Inventors: Erik Schulze zur Wiesche, Hamburg (DE); Rene Krohn, Norderstedt (DE)

(73) Assignee: Henkel AG & Co. KGaA, Duesseldorf (DE)

(21) Appl. No.: 15/151,659

(22) Filed: May 11, 2016

Related U.S. Application Data
(63) Continuation of application No. PCT/EP2014/073668, filed on Nov. 4, 2014.

(30) Foreign Application Priority Data
Dec. 5, 2013 (DE) 10 2013 224 999.3

(51) Int. Cl. A61K 8/81; A61K 8/58; A61K 8/49; A61Q 5/00

(52) U.S. Cl. CPC A61K 8/8152; A61Q 5/002; A61K 8/585; A61K 8/8147; A61K 8/4946

(57) ABSTRACT

The invention relates to a hair treatment agent including 0.1 to 20 weight percent of at least one silicone including groups of the formula (I) as defined herein; 0.1 to 20 weight percent of at least one copolymer that includes monomer units of the formulas (V) and (VI) as defined herein; and 0.5 to 5 weight percent of 2-phenylbenzimidazole-5-sulfonic acid. The hair treatment agent according to the invention has an improved protective effect against UV radiation and has an improving effect on hair treated therewith with regard to combability, gloss, elasticity, brittleness, and maximum tensile strength.

### FIELD OF THE INVENTION

The present invention generally relates to hair treatment agents including specifically substituted silicone(s), and the use of these agents for the cleaning and/or care of hair.

### BACKGROUND OF THE INVENTION

Care agents for keratinic fibers influence the natural structure and the properties of the hair. Thus, for example, the wet and dry combability of the hair, the hold, and the fullness of the hair may be optimized, or the hair may be protected from increased split ends, following such treatments. Therefore, it has been common for quite some time to subject the hair to a special aftertreatment in which the hair is treated, typically in the form of a rinse, with particular active substances, for example quaternary ammonium salts or specific polymers. As a result of this treatment, the combability, the hold, and the fullness of the hair are improved and the frequency of split ends is reduced, depending on the formulation.

For example, the structure of the hair sustains lasting damage from UV light due to intensive use of tanning salons or extensive physical recreational activity. This damage is manifested in the hair by a loss in the elasticity, for example. Therefore, it is not surprising that the number of consumers with sensitive, less elastic, more brittle, and irritable skin as well as hair with impaired combability, gloss, elasticity, brittleness, and maximum tensile strength is increasing dramatically.

Another problem is found with dyed hair. If the dyes formed or used directly in the course of the color formation have greatly different fastness properties (for example, UV stability, perspiration fastness, wash fastness, etc.), a noticeable and therefore undesirable color shift may occur over time. This phenomenon is even more apparent when the hairstyle has hairs or hair zones with different degrees of damage. One example is long hair in which tips of the hair that experience long-term exposure to all possible environmental influences are generally damaged much more than the hair zones that have grown out relatively recently.

For this reason, there is a need to provide care products which have a great protective effect against fading of the natural or dyed hair color and against the damage to the hair structure by UV radiation.

Therefore, it has been customary for quite some time in skin cosmetics as well as hair cosmetics to incorporate UV filters into the agents. According to European law, UV filters are defined according to their intended use. Accordingly, they are “substances which are exclusively or mainly intended to protect the skin against certain UV radiation by absorbing, reflecting, or scattering certain UV radiation.” Regardless of whether organic or inorganic, so-called chemical or physical, natural or synthetic UV filters are involved, all UV filters are classified according to this definition. Only UV filters which are listed in the Cosmetics Regulation may be used. Since the individual substances generally do not provide protection over the entire UV spectrum, multiple substances are usually combined.

In addition, synergistic effects typically result from the combination of UV filters.

While UV protection in skin care products has been developed extensively with respect to formulations, in hair care products less complex systems are used for the “dead” matter of hair. In addition, the quantities in which filter substances can be incorporated are generally smaller, partly for formulation reasons, and partly for cost reasons. In order to still be able to provide effective UV protection, it is desirable to improve the protective effect of known, less expensive UV filters, and also to impart the agents in question with positive effects with regard to combability, gloss, elasticity, brittleness, and maximum tensile strength of the hair treated with same.

Furthermore, other desirable features and characteristics of the present invention will become apparent from the subsequent detailed description of the invention and the appended claims, taken in conjunction with this background of the invention.

### BRIEF SUMMARY OF THE INVENTION

It has now been found that agents including 2-phenylbenzimidazole-5-sulfonic acid may be greatly improved with regard to the set of tasks mentioned above when certain acrylate polymers and substituted silicones are used.

Such a hair treatment agent includes, in each case based on its weight, 0.1 to 20% by weight of at least one silicone including groups of formula (I)

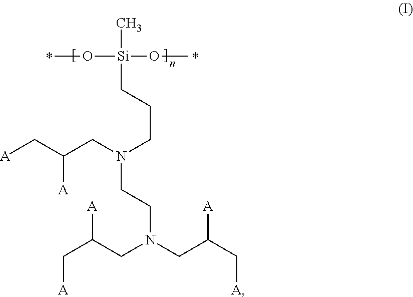

in which n stands for values from 1 to 1000, A stands for a group

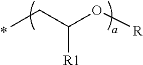

in which R1 stands for —H or —CH₃ and R stands for a straight-chain or branched alkyl moiety including 8 to 24 C atoms, and a stands for an integer from 1 to 20; 0.1 to 20% by weight of at least one copolymer including monomer units of formulas (V) and (VI)

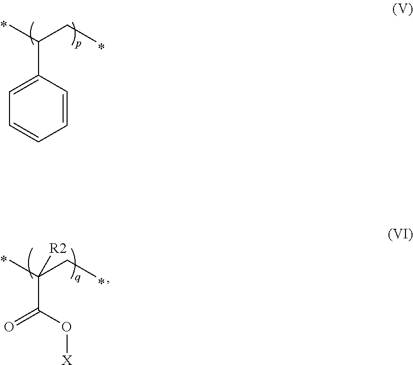

in which R2 stands for —H or —CH₃ and X stands for H, an alkali metal cation, or a straight-chain or branched alkyl moiety including 1 to 24 C atoms; and 0.5 to 5% by weight of 2-phenylbenzimidazole-5-sulfonic acid.

Silicones used to accomplish these improvements include groups of formula (I)

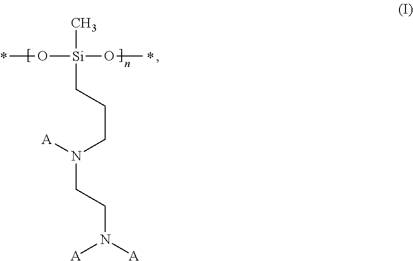

in which n stands for values from 1 to 1000, A stands for a group

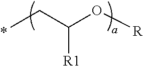

in which R1 stands for —H or —CH₃ and R stands for a straight-chain or branched alkyl moiety including 8 to 24 C atoms, and a stands for an integer from 1 to 20, and are particularly used for reducing the damage to the hair structure by external influences, in particular by UV radiation.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject matter of the present invention is a hair treatment agent which includes, in each case based on its weight,
a) 0.1 to 20% by weight of at least one silicone including groups of formula (I)

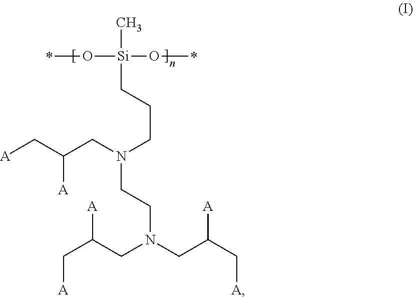

in which
n stands for values from 1 to 1000,
A stands for a group

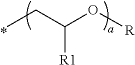

in which R1 stands for —H or —CH₃ and R stands for a straight-chain or branched alkyl moiety including 8 to 24 C atoms, and a stands for an integer from 1 to 20;
b) 0.1 to 20% by weight of at least one copolymer including monomer units of formulas (V) and (VI)

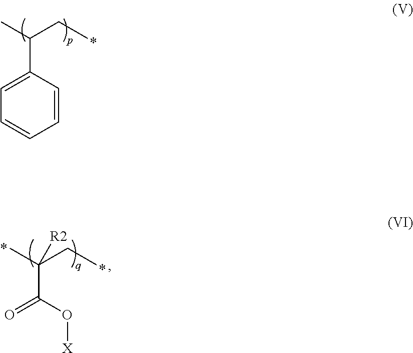

in which R2 stands for —H or —CH₃ and X stands for H, an alkali metal cation, or a straight-chain or branched alkyl moiety including 1 to 24 C atoms;
c) 0.5 to 5% by weight of 2-phenylbenzimidazole-5-sulfonic acid.

Within the meaning of the present invention, examples of hair treatment agents are hair shampoos, hair conditioners, conditioning shampoos, hair sprays, hair rinses, hair masks, hair packs, hair tonics, permanent wave fixing solutions, hair-coloring shampoos, hair colorants, hair fixatives, hair setting agents, hairstyling preparations, blow-dry lotions, mousses, hair gels, hair waxes, or combinations thereof. In view of the fact that men often hesitate to apply a number of different agents and/or carry out multiple application steps, agents according to the invention are preferably those agents which men use anyway. Preferred agents according to the invention are therefore shampoos, conditioning agents, or hair tonics.

The compositions of the invention impart improved properties to the keratinic fibers which are treated with same; these properties (lightness, softness, ease of untangling, natural feel and buoyancy of the hair, luminosity, for example) last even under intensive UV irradiation, and in addition the effects are persistent and long-lasting. In particular, these effects withstand multiple shampooings.

The agents according to the invention include as the first essential ingredient 0.1 to 20% by weight of at least one silicone including groups of formula (I). In formula (I), n stands for values of 1 to 1000, wherein values 1 to 40, preferably 1 to 30, more preferably 1 to 20, and in particular for the values 2, 3, 4, 5, 6, 7, 8, 9, or 10, are preferred.

In formula (I), A stands for a group

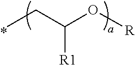

in which R1 stands for —H or —CH₃ and R stands for a straight-chain or branched alkyl moiety including 8 to 24 C atoms, and a stands for an integer from 1 to 20;
R1 is particularly preferably —H.
A particularly good UV protective effect is achieved when the moiety R stands for

—(CH₂)ₖ—CH₃, where k=7, 8, 9, 10, 11, 12, 13, 14, 15, 16, 17

—CH(CH₂CH₃)—(CH₂)₅.

Particularly preferred hair treatment agents according to the invention are characterized in that they include 0.1 to 20% by weight, preferably 0.25 to 15% by weight, more preferably 0.5 to 10% by weight, even more preferably 0.75 to 7.5% by weight, and in particular 1 to 5% by weight, of at least one silicone including groups of formula (Ia)

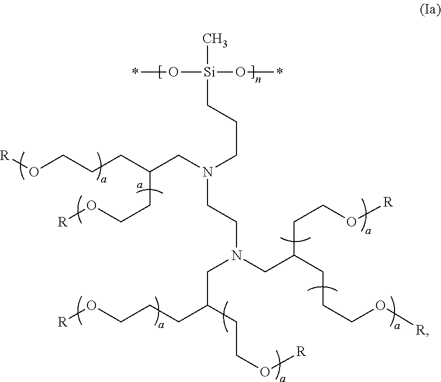

in which R is selected from

—(CH₂)ₖ—CH₃, where k=7, 8, 9, 10, 11, 12, 13, 14, 15, 16, 17

—CH(CH₂CH₃)—(CH₂)₅

and a stands for an integer from 1 to 20.

The silicones used according to the invention may be composed completely of groups of formula (I) or (Ia), wherein —CH₃, —O—CH₃, or —OH are preferred as terminal groups.

However, it is preferred that the silicones used according to the invention include further groups, in particular those of formula

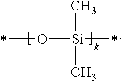

In addition, co-silicones made up of units of the above-mentioned formula and units of formula (I) or (Ia) may have —CH₃, —O—CH₃, or —OH as terminal groups. The units may be present in such co-silicones as a block or statistically distributed.

Particularly preferred hair treatment agents according to the invention are characterized in that they include 0.1 to 20% by weight, preferably 0.25 to 15% by weight, more preferably 0.5 to 10% by weight, even more preferably 0.75 to 7.5% by weight, and in particular 1 to 5% by weight, of at least one silicone of formula (II):

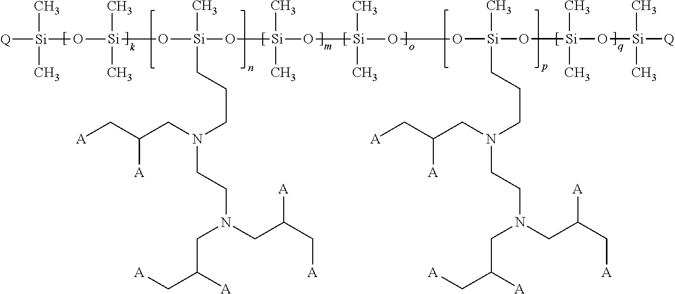

in which

A stands for a group

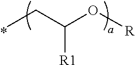

in which R1 stands for —H or —CH₃ and R stands for a straight-chain or branched alkyl moiety including 8 to 24 C atoms, and a stands for an integer from 1 to 20;

Q stands for —OH, —CH₃, or —OCH₃;

the indices k, n, m, o, p, q stand for integers from 0 to 1000, with the condition that n+p>0 and k+m+o+q>0,

wherein the units having the indices k, n, m, o, p, q may be present in the molecule as a block or statistically distributed.

Also for the silicones of formula (II), A stands for a group

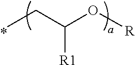

in which R1 stands for —H or —CH₃ and R stands for a straight-chain or branched alkyl moiety including 8 to 24 C atoms, and a stands for an integer from 1 to 20;
R1 is particularly preferably —H.
A particularly good UV protective effect is achieved when the moiety R stands for

—(CH₂)ₖ—CH₃, where k=7, 8, 9, 10, 11, 12, 13, 14, 15, 16, 17

—CH(CH₂CH₃)—(CH₂)₅.

Further preferred hair treatment agents according to the invention are therefore characterized in that they include 0.1 to 20% by weight, preferably 0.25 to 15% by weight, more preferably 0.5 to 10% by weight, even more preferably 0.75 to 7.5% by weight, and in particular 1 to 5% by weight, of at least one silicone of formula (III):

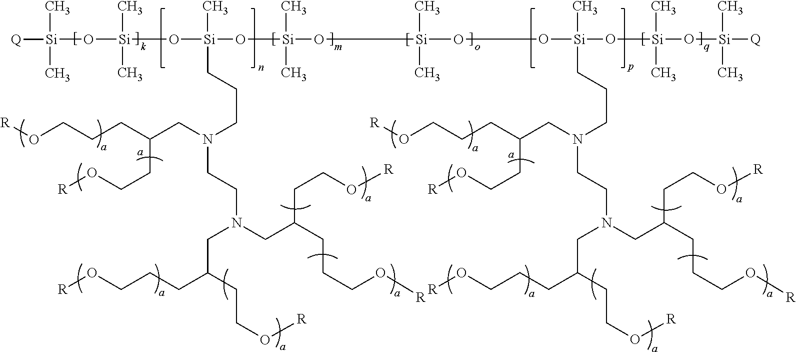

in which
R is selected from

—(CH₂)ₖ—CH₃, where k=7, 8, 9, 10, 11, 12, 13, 14, 15, 16, 17

—CH(CH₂CH₃)—(CH₂)₅

a stands for an integer from 1 to 20;
Q stands for —OH, —CH₃, or —OCH₃;
the indices k, n, m, o, p, q stand for integers from 0 to 1000, with the condition that

n+p>0 and k+m+o+q>0,

wherein the units having the indices k, n, m, o, p, q may be present in the molecule as a block or statistically distributed.

It is very particularly preferred that the index a stands for the number 9, and R is a straight-chain alkyl moiety including 13 carbon atoms. In particular, preferred hair treatment agents are therefore characterized in that they include 0.1 to 20% by weight, preferably 0.25 to 15% by weight, more preferably 0.5 to 10% by weight, even more preferably 0.75 to 7.5% by weight, and in particular 1 to 5% by weight, of at least one silicone of formula (IV):

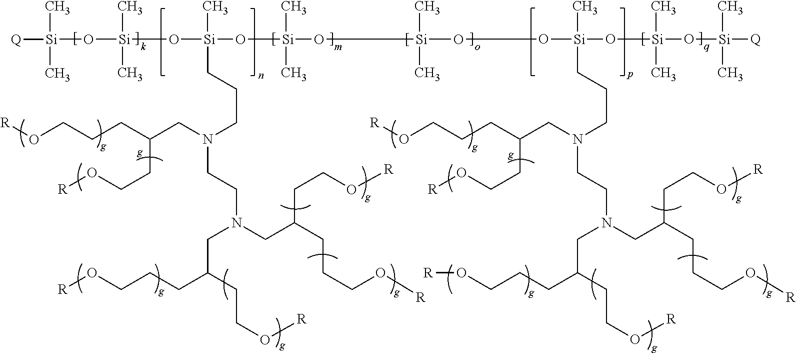

in which
R stands for —(CH₂)₁₂—CH₃ and Q stands for —OH or —OCH₃;
Q stands for —OH, —CH₃, or —OCH₃;
the indices k, n, m, o, p, q stand for integers from 0 to 1000, with the condition that

n+p>0 and k+m+o+q>0,

wherein the units having the indices k, n, m, o, p, q may be present in the molecule as a block or statistically distributed.

In formulas (II), (III), and (IV), Q stands for —OH, —CH₃, or —OCH₃. In all three formulas the two Q's do not have to be identical; rather, the termination at one end may be different than at the other end, the following combinations being preferred:

| “Left” terminal group Q | “Right” terminal group Q |
|---|---|
| —OH | —OH |
| —CH₃ | —OH |
| —OCH₃ | —OH |
| —OH | —CH₃ |
| —CH₃ | —CH₃ |
| —OCH₃ | —CH₃ |
| —OH | —OCH₃ |
| —CH₃ | —OCH₃ |
| —OCH₃ | —OCH₃ |

It is particularly preferred for the two groups at the respective chain end to be identical. In particular, Q preferably stands for —OH or —OCH₃. The silicones of formulas (IIa), (IIb), (IIIa), (IIIb), (IVa), and (IVb) represent particularly preferred embodiments:

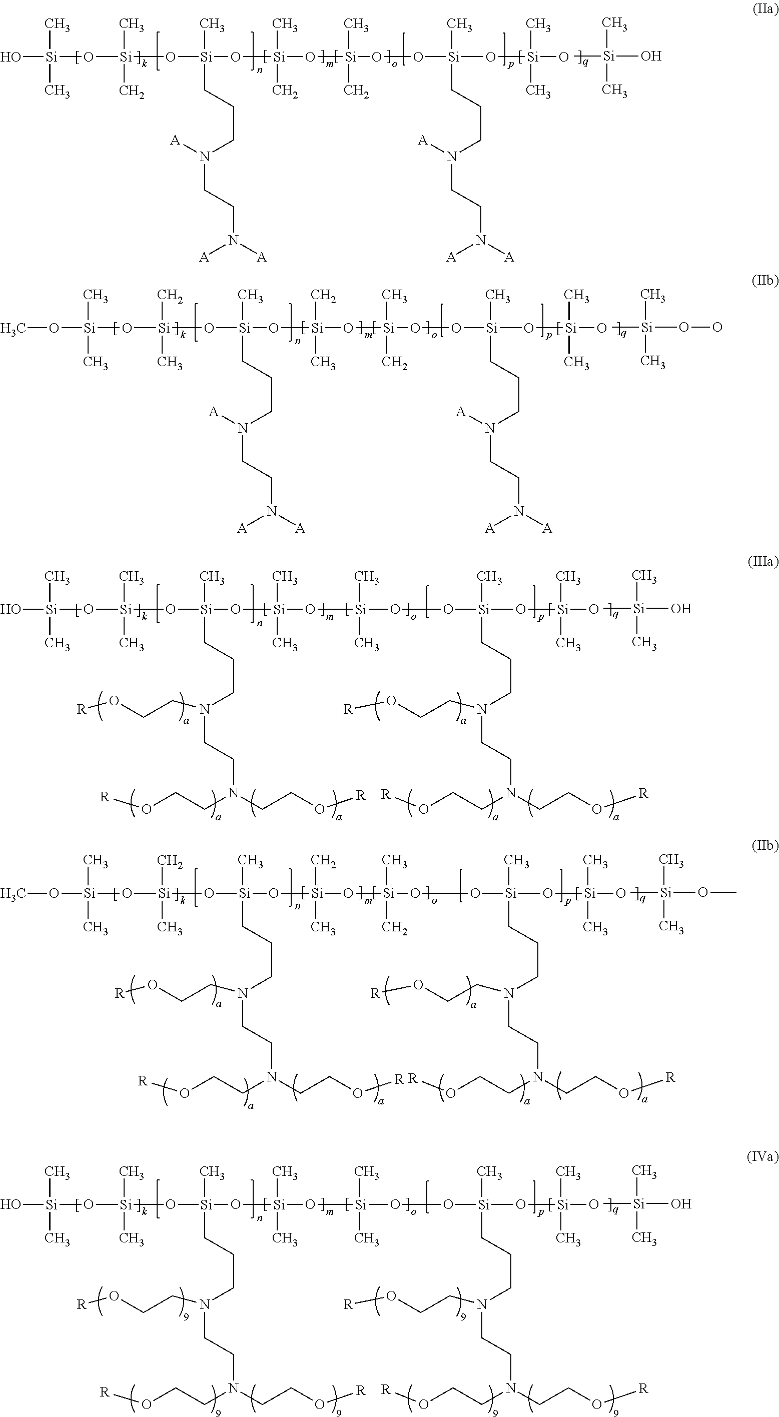

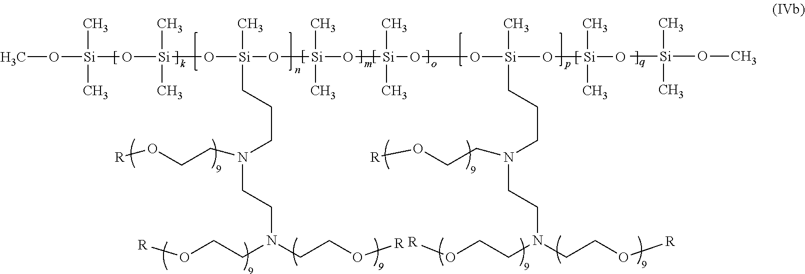

The average molecular weights of the silicones a) included in the agents according to the invention are preferably 2,000 to 300,000 Dalton, even more preferably 5,000 to 200,000 Dalton, in particular 10,000 to 150,000 Dalton. Hair treatment agents are preferred in which the weight-average molar mass of the silicone of formula (IV) included therein is in the range of 10,000 to 150,000 gmol⁻¹, preferably in the range of 50,000 to 130,000 gmol⁻¹.

The average molecular weights of amino-substituted silicones are measurable, for example, by gel permeation chromatography (GPC) in polystyrene at room temperature. Styragel columns may be selected as columns, THF may be selected as the eluent, and 1 mL/min may be selected as the flow rate. The detection preferably takes place by means of refractometry and UV meters.

As a further component, the agents according to the invention include 0.1 to 20% by weight of at least one copolymer which has monomer units of formulas (V) and (VI)

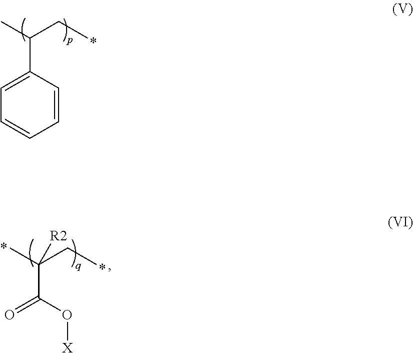

in which R2 stands for —H or —CH₃ and X stands for —H, an alkali metal cation, or a straight-chain or branched alkyl moiety including 1 to 24 C atoms.

The copolymers may be composed only of monomers of formulas (V) and (VI), but may also include other monomer structural units. With regard to the protective effect against damage to the hair structure, it has proven to be preferable when at least 50% by weight, preferably at least 60% by weight, more preferably at least 70% by weight, even more preferably at least 80% by weight, particularly preferably at least 90% by weight, and in particular at least 99% by weight, of the copolymers are composed of monomers of formulas (V) and (VI).

The monomer to be associated with the structural unit (V) is styrene, and the structural unit (VI) may be derived from acrylic acid (R2=—H, X=—H), methacrylic acid (R2=—CH₃, X=—H), the salts thereof (X=Na⁺, K⁺, etc.) and/or the esters thereof (X=alkyl). The copolymers may include a single type of monomer unit (VI) or a number of various monomer units (VI, VI′, VI″, etc.). An example for the first embodiment would be copolymers of styrene and acrylic acid, and an example for the second embodiment would be copolymers of styrene, acrylic acid, and methacrylate.

For the copolymers which include only monomer units of formulas (V) and (VI), the following copolymers are preferred:
copolymers of styrene and acrylic acid
copolymers of styrene and methacrylic acid
copolymers of styrene and sodium acrylate
copolymers of styrene and sodium methacrylate
copolymers of styrene and potassium acrylate
copolymers of styrene and potassium methacrylate
copolymers of styrene and acrylic acid methyl ester
copolymers of styrene and methacrylic acid methyl ester
copolymers of styrene and acrylic acid ethyl ester
copolymers of styrene and methacrylic acid ethyl ester
copolymers of styrene and acrylic acid propyl ester
copolymers of styrene and methacrylic acid propyl ester
copolymers of styrene and acrylic acid isopropyl ester
copolymers of styrene and methacrylic acid isopropyl ester
copolymers of styrene and acrylic acid (n-butyl) ester
copolymers of styrene and methacrylic acid (n-butyl) ester
copolymers of styrene and acrylic acid and methacrylic acid
copolymers of styrene and acrylic acid and sodium acrylate
copolymers of styrene and acrylic acid and sodium methacrylate
copolymers of styrene and acrylic acid and acrylic acid methyl ester
copolymers of styrene and acrylic acid and methacrylic acid methyl ester
copolymers of styrene and acrylic acid and acrylic acid ethyl ester
copolymers of styrene and acrylic acid and methacrylic acid ethyl ester
copolymers of styrene and acrylic acid and acrylic acid (n-butyl) ester
copolymers of styrene and acrylic acid and methacrylic acid (n-butyl) ester
copolymers of styrene and methacrylic acid and sodium acrylate
copolymers of styrene and methacrylic acid and sodium methacrylate
copolymers of styrene and methacrylic acid and acrylic acid methyl ester
copolymers of styrene and methacrylic acid and methacrylic acid methyl ester
copolymers of styrene and methacrylic acid and acrylic acid ethyl ester
copolymers of styrene and methacrylic acid and methacrylic acid ethyl ester
copolymers of styrene and methacrylic acid and acrylic acid (n-butyl) ester
copolymers of styrene and methacrylic acid and methacrylic acid (n-butyl) ester
copolymers of styrene and acrylic acid and methacrylic acid and sodium acrylate
copolymers of styrene and acrylic acid and methacrylic acid and sodium methacrylate
copolymers of styrene and acrylic acid and methacrylic acid and acrylic acid methyl ester
copolymers of styrene and acrylic acid and methacrylic acid and methacrylic acid methyl ester
copolymers of styrene and acrylic acid and methacrylic acid and acrylic acid ethyl ester
copolymers of styrene and acrylic acid and methacrylic acid and methacrylic acid ethyl ester
copolymers of styrene and acrylic acid and methacrylic acid and acrylic acid (n-butyl) ester
copolymers of styrene and acrylic acid and methacrylic acid and methacrylic acid (n-butyl) ester.

In particular, preferred hair treatment agents are therefore characterized in that they include 0.5 to 17.5% by weight, preferably 1 to 15% by weight, more preferably 1.5 to 12.5% by weight, particularly preferably 2 to 10% by weight, and in particular 3 to 8% by weight, of at least one copolymer having monomer units of formulas (V) and (VIa)

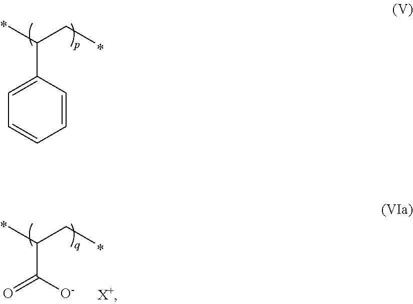

in which X stands for H⁺ or Na⁺.

As a further component, the agents according to the invention include 0.5 to 5% by weight of 2-phenylbenzimidazole-5-sulfonic acid. Particularly preferred hair treatment agents according to the invention are characterized in that they include 0.55 to 4.5% by weight, preferably 0.6 to 4% by weight, more preferably 0.65 to 3.5% by weight, particularly preferably 0.7 to 3% by weight, and in particular 0.75 to 2.5% by weight, of 2-phenylbenzimidazole-5-sulfonic acid.

It has proven to be preferable when the ingredients a), b), and c) are used in certain quantity ratios relative to one another. The UV protective effect is optimally intensified in this way. Hair treatment agents which are preferred according to the invention are characterized in that the weight ratio of ingredients b) to a) is in the range of 1:50 to 50:1, preferably in the range of 1:10 to 25:1, more preferably in the range of 1:1 to 5:1, particularly preferably in the range of 1.5:1 to 4:1, and in particular in the range of 1.75:1 to 2.5:1.

Further preferred hair treatment agents according to the invention are characterized in that the weight ratio of ingredients a) to c) is in the range of 1:50 to 50:1, preferably in the range of 1:10 to 25:1, more preferably in the range of 1:5 to 5:1, particularly preferably in the range of 1.5:1 to 4:1, and in particular in the range of 1.1:1 to 1:1.1.

The agents according to the invention each include further essential ingredients, depending on the intended use. Cleaning or care compositions such as shampoos or conditioners, for example, include at least one surfactant; depending on the field of application, surface-active substances are referred to as surfactants or as emulsifiers, and are selected from anionic, cationic, zwitterionic, ampholytic, and nonionic surfactants and emulsifiers.

Hair treatment agents which are preferred according to the invention are characterized in that, based on their weight, they include 0.5 to 70% by weight, preferably 1 to 60% by weight, and in particular 5 to 25% by weight, of anionic and/or nonionic and/or cationic and/or amphoteric surfactant(s).

For the compositions according to the invention, all anionic surface-active substances which are suitable for use on the human body are appropriate as anionic surfactants and emulsifiers. These are characterized by a water-solubilizing anionic group, for example a carboxylate, sulfate, sulfonate, or phosphate group, and a lipophilic alkyl group including approximately 8 to 30 C atoms. In addition, glycol ether or polyglycol ether groups, ester groups, ether groups, and amide groups as well as hydroxyl groups may be included in the molecule.

Zwitterionic surfactants and emulsifiers refer to those surface-active compounds which bear at least one quaternary ammonium group and at least one —COO⁽⁻⁾ or —SO₃⁽⁻⁾ group in the molecule. Particularly suitable zwitterionic surfactants and emulsifiers are the so-called betaines such as N-alkyl-N,N-dimethylammonium glycinates, for example coco-alkyldimethylammonium glycinate; N-acylaminopropyl-N,N-dimethylammonium glycinates, for example coco-acylaminopropyldimethylammonium glycinate, and 2-alkyl-3-carboxymethyl-3-hydroxyethylimidazolines in each case including 8 to 18 C atoms in the alkyl or acyl group; and coco-acylaminoethylhydroxyethylcarboxymethyl glycinate. One preferred zwitterionic surfactant is the fatty acid amide derivative known by the INCI name Cocamidopropyl Betaine.

Ampholytic surfactants and emulsifiers are understood to mean those surface-active compounds which in addition to a C₈-C₂₄ alkyl or acyl group include at least one free amino group and at least one —COOH or —SO₃H group and which are capable of forming internal salts. Examples of suitable ampholytic surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids in each case including approximately 8 to 24 C atoms in the alkyl group. Particularly preferred ampholytic surfactants are N-coco-alkylaminopropionate, coco-acylaminoethylaminopropionate, and C₁₂-C₁₈ acylsarcosine.

Cationic surfactants of the quaternary ammonium compound, esterquat, and amidoamine types are usable according to the invention. Preferred quaternary ammonium compounds are ammonium halides, in particular chlorides and bromides, such as alkyltrimethylammonium chlorides, dialkyldimethylammonium chlorides, and trialkylmethylammonium chlorides. The long alkyl chains of these surfactants preferably include 10 to 18 carbon atoms, such as in cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride, for example. Further preferred cationic surfactants are the imidazolium compounds known by the INCI names Quaternium-27 and Quaternium-83.

Particularly preferred hair treatment agents according to the invention are characterized in that as a cationic care substance, based on their weight, they include 0.05 to 7.5% by weight, preferably 0.1 to 5% by weight, particularly preferably 0.2 to 3.5% by weight, and in particular 0.25 to 2.5% by weight, of cationic surfactant(s) from the group of quaternary ammonium compounds and/or esterquats and/or amidoamines.

The agents according to the invention may particularly preferably include one or more amino acids as a further ingredient. Amino acids which are particularly preferably usable according to the invention come from the group comprising glycine, alanine, valine, leucine, isoleucine, phenylalanine, tyrosine, tryptophan, proline, aspartic acid, glutaminic acid, asparagine, glutamine, serine, threonine, cysteine, methionine, lysine, arginine, histidine, β-alanine, 4-aminobutyric acid (GABA), betaine, L-cystine (L-Cyss), L-carnitine, L-citrulline, L-theanine, 3′,4′-dihydroxy-L-phenylalanine (L-DOPA), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutaminic acid), L-phosphoserine, creatine, 3-methyl-L-histidine, and L-ornithine, wherein the individual amino acids as well as mixtures may be used.

Preferred agents according to the invention include one or more amino acids in fairly narrow quantity ranges. Cosmetic agents which are preferred here according to the invention are characterized in that as a care substance, based on their weight, they include 0.01 to 5% by weight, preferably 0.02 to 2.5% by weight, particularly preferably 0.05 to 1.5% by weight, more preferably 0.075 to 1% by weight, and in particular 0.1 to 0.25% by weight, of amino acid(s), preferably from the group comprising glycine and/or alanine and/or valine and/or lysine and/or leucine and/or threonine.

Of course, the silicones used according to the invention having structural units of formula (I) may be used together with other conventional silicones.

Agents preferred according to the invention are characterized in that they include at least one further silicone.

Of course, mixtures of the above-mentioned silicones may also be included in the agents according to the invention.

Preferred silicones which are usable according to the invention have viscosities of 0.2 to 2 mm²s⁻¹ at 20° C., wherein silicones having viscosities of 0.5 to 1 mm²s⁻¹ are particularly preferred.

Particularly preferred agents according to the invention include one or more aminofunctional silicones.

Preferred agents according to the invention are characterized in that they include an aminofunctional silicone of formula (Si-II)

R′ₐG₃₋ₐ-Si(OSiG₂)ₙ-(OSiGbR′_{2-b})ₘ—O—SiG₃₋ₐ-R′ₐ (Si-II),

where the terms have the following meanings:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a stands for a number between 0 and 3, in particular 0,
b stands for a number between 0 and 1, in particular 1,
m and n are numbers whose sum (m+n) is between 1 and 2000, preferably between 50 and 150, where n preferably assumes values of 0 to 1999 and in particular 49 to 149, and m preferably assumes values of 1 to 2000, in particular 1 to 10,
R′ is a monovalent moiety selected from
   -Q-N(R″)—CH₂—CH₂—N(R″)₂
   -Q-N(R″)₂
   -Q-N⁺(R″)₃A⁻
   -Q-N⁺H(R″)₂A⁻
   -Q-N⁺H₂(R″)A⁻
   -Q-N(R″)—CH₂—CH₂—N⁺R″H₂A⁻,
      wherein each Q stands for a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
      R″ stands for moieties, which are the same or different, from the group comprising —H, -phenyl, -benzyl, —CH₂—CH(CH₃)Ph, C₁₋₂₀ alkyl moieties, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion which is preferably selected from chloride, bromide, iodide, or methosulfate.

Particularly preferred agents according to the invention are characterized in that they include at least one aminofunctional silicone of formula (Si-IIa)

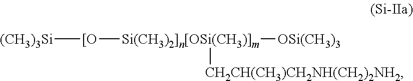

where m and n are numbers whose sum (m+n) is between 1 and 2000, preferably between 50 and 150, where n preferably assumes values of 0 to 1999 and in particular 49 to 149, and m preferably assumes values of 1 to 2000, in particular 1 to 10.

These silicones are referred to as Trimethylsilylamodimethicones according to the INCI declaration.

Also particularly preferred according to the invention are agents which include an aminofunctional silicone of formula (Si-IIb)

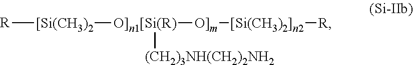

where R stands for —OH, —O—CH₃, or a —CH₃ group, and m, n1, and n2 are numbers whose sum (m+n1+n2) is between 1 and 2000, preferably between 50 and 150, where the sum (n1+n2) preferably assumes values of 0 to 1999 and in particular 49 to 149, and m preferably assumes values of 1 to 2000, in particular 1 to 10.

These silicones are referred to as Amodimethicones according to the INCI declaration.

Regardless of which aminofunctional silicones are used, agents according to the invention are preferred which include an aminofunctional silicone whose amine value is above 0.25 meq/g, preferably above 0.3 meq/g, and in particular above 0.4 meq/g. The amine value stands for the milliequivalents of amine per gram of the aminofunctional silicone. It may be determined by titration, and is also expressed in the units mg KOH/g.

Agents preferred according to the invention are characterized in that, based on their weight, they include 0.01 to 10% by weight, preferably 0.1 to 8% by weight, particularly preferably 0.25 to 7.5% by weight, and in particular 0.5 to 5% by weight, of aminofunctional silicone(s).

The silicones described above have a backbone which is built from —Si—O—Si units. Of course, these Si—O—Si units may also be interrupted by carbon chains. Such molecules are obtainable by chain lengthening reactions, and are preferably used in the form of silicone-in-water emulsions.

Agents likewise preferred according to the invention are characterized in that they include at least one silicone of formula (Si-IV)

R₃Si—[O—SiR₂]ₓ—(CH₂)ₙ—[O—SiR₂]_{y}—O—SiR₃ (Si-IV),

in which R stands for moieties, which are the same or different, from the group comprising —H, -phenyl, -benzyl, —CH₂—CH(CH₃)Ph, C₁₋₂₀ alkyl moieties, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y each stand for a number from 0 to 200, preferably from 0 to 10, more preferably from 0 to 7, and in particular 0, 1, 2, 3, 4, 5 or 6, and n stands for a number from 0 to 10, preferably from 1 to 8, and in particular for 2, 3, 4, 5, 6.

The silicones are preferably water-soluble. Agents preferred according to the invention are characterized in that they include at least one water-soluble silicone.

It has been shown that certain proteolipids even further intensify the effect of the combination according to the invention, and even further improve the prevention of damage to the hair structure by UV radiation. As a further ingredient, the agents according to the invention may include at least one proteolipid of formula (P-I)

R′—X—R″ (P-I),

in which
R′ stands for a straight-chain or branched, saturated or unsaturated hydrocarbon moiety including 11 to 24 carbon atoms,
R″ means a protein, a peptide, or a protein hydrolysate,
X stands for —C(O)O— or —N⁺(R^{III}₂)R^{IV}— or —N(R^{III})R^{IV}— or —C(O)—N(R^{V})R^{VI}—,
R^{III} means —(CH₂)ₓ—CH₃, where x=0-22 and
R^{IV} means —CH₂—CH(OH)—CH₂— or —(CH₂)ₓ—, where x=0-22;

R^{V} and R^{V} independently stand for —H or —(CH₂)ₓ—CH₃, where x=0-22;

with the condition that R″ stands for keratin or a keratin hydrolysate when X stands for —C(O)O—.

The proteolipids are preferably used within certain quantities in the agents according to the invention. Preferred cosmetic agents according to the invention, based on their weight, include 0.01 to 10% by weight, preferably 0.02 to 5% by weight, particularly preferably 0.05 to 2.5% by weight, more preferably 0.1 to 1% by weight, and in particular 0.15 to 0.5% by weight, of proteolipid(s). The moiety R″ in formula (P-I) stands for a peptide, a protein, or a protein hydrolysate. R″ is selected from the group comprising keratin or keratin hydrolysate when X=—C(O)O—.

Preferred moieties R″ are oligopetides having at least one amino acid sequence Glu-Glu-Glu

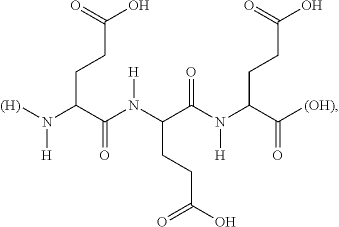

wherein the amino group may be present in the free or protonated form, and the carboxy groups may be present in the free or deprotonated form.

In these formulas as well as in all the formulas below, the hydrogen atom in parentheses in the amino group, and also the hydroxy group in parentheses in the acid function, mean that the groups in question may be present as such (which is then an oligopeptide having the applicable number of amino acids as illustrated (in the present formula, 3)), or that the amino acid sequence is present in an oligopeptide which includes even further amino acids; depending on where the further amino acid(s) is/are bonded, the components in parentheses in the above-mentioned formula are replaced by the further amino acid moiety/moieties.

Within the meaning of the present patent application, oligopeptides are condensation products of amino acids which include at least 3 and at most 25 amino acids, and which are linked by peptide bonds in the manner of an acid amide.

In hair treatment agents of the above-described embodiment which are preferred according to the invention, the oligopeptide (the moiety R″) includes 5 to 15 amino acids, preferably 6 to 13 amino acids, particularly preferably 7 to 12 amino acids, and in particular 8, 9, or 10 amino acids.

The molar mass of the proteolipid included in the agents according to the invention may vary, depending on whether further amino acids are bonded to the Glu-Glu-Glu sequence, and depending on the type of these amino acids and as a function of the selection of the moieties R′ and optionally R^{III} and R^{IV}. Cosmetic agents preferred according to the invention are characterized in that the proteolipid has a molar mass of 1,000 to 30,000 Da, preferably 1,250 to 25,000 Da, particularly preferably 1,500 to 20,000 Da, and in particular 2,000 to 15,000 Da.

Preferably used as the moiety R″ are oligopeptides which are not composed solely of the three glutaminic acids, but which instead include further amino acids that are bonded to this sequence. These further amino acids are preferably selected from certain amino acids, while certain other representatives are less preferred according to the invention.

Thus, it is preferred when the moiety R″ of the proteolipids used in the agents according to the invention includes no methionine. It is further preferred when the moiety R″ of the proteolipids used in the agents according to the invention includes no cysteine and/or cystine.

It is further preferred when the moiety R″ of the proteolipids used in the agents according to the invention includes no aspartic acid and/or asparagine. It is further preferred when the moiety R″ of the proteolipids used in the agents according to the invention includes no serine and/or threonine.

On the other hand, it is preferred when the moiety R″ of the proteolipids used in the agents according to the invention includes tyrosine. It is further preferred when the moiety R″ of the proteolipids used in the agents according to the invention includes leucine. It is further preferred when the moiety R″ of the proteolipids used in the agents according to the invention includes isoleucine. It is further preferred when the moiety R″ of the proteolipids used in the agents according to the invention includes arginine. It is further preferred when the moiety R″ of the proteolipids used in the agents according to the invention includes valine.

Oligopeptides which are particularly preferred as the moiety R″ and amino acid sequences which are included in the preferred oligopeptides are described below:

One particularly preferred oligopeptide additionally includes tyrosine, which is preferably bonded via its acid function to the Glu-Glu-Glu sequence. Cosmetic agents preferred according to the invention are therefore characterized in that the oligopeptide included in the proteolipids of formula (I) as the moiety R″ has at least one amino acid sequence Tyr-Glu-Glu-Glu, wherein the amino group may be present in the free or protonated form, and the carboxy groups may be present in the free or deprotonated form.

Another particularly preferred oligopeptide additionally includes isoleucine, which is preferably bonded via its acid function to the Glu-Glu-Glu sequence. Cosmetic agents preferred according to the invention are therefore characterized in that the oligopeptide included in the proteolipids of formula (I) as the moiety R″ has at least one amino acid sequence Glu-Glu-Glu-Ile, wherein the amino group may be present in the free or protonated form, and the carboxy groups may be present in the free or deprotonated form.

Oligopeptides which include both of the amino acids mentioned above (tyrosine and isoleucine) are preferred according to the invention. Hair treatment agents according to the invention are particularly preferred in which the oligopeptide included in the proteolipids of formula (I) as the moiety R″ has at least one amino acid sequence Tyr-Glu-Glu-Glu-Ile, wherein the amino group may be present in the free or protonated form, and the carboxy groups may be present in the free or deprotonated form.

Further preferred oligopeptides additionally include arginine, which is preferably present bonded to isoleucine. Cosmetic agents preferred according to the invention are therefore characterized in that the oligopeptide included in the proteolipids of formula (I) as the moiety R″ has at least one amino acid sequence Tyr-GIu-GIu-GIu-IIe-Arg, wherein the amino groups may be present in the free or protonated form, and the carboxy groups may be present in the free or deprotonated form.

Even further preferred oligopeptides additionally include valine, which is preferably present bonded to the arginine. Cosmetic agents further preferred according to the invention are therefore characterized in that the oligopeptide included in the proteolipids of formula (I) as the moiety R″ has at least one amino acid sequence Tyr-Glu-Glu-Glu-Ile-Arg-Val, wherein the amino groups may be present in the free or protonated form, and the carboxy groups may be present in the free or deprotonated form.

Even further preferred oligopeptides additionally include leucine, which is preferably present bonded to valine. Cosmetic agents further preferred according to the invention are therefore characterized in that the oligopeptide included in the proteolipids of formula (I) as the moiety R″ has at least one amino acid sequence Tyr-Glu-Glu-Glu-Ile-Arg-Val-Leu, wherein the amino groups may be present in the free or protonated form, and the carboxy groups may be present in the free or deprotonated form.

In particular, preferred oligopeptides additionally include leucine which is preferably present bonded to the tyrosine. Cosmetic agents further preferred according to the invention are therefore characterized in that the oligopeptide included in the proteolipids of formula (I) as the moiety R″ has at least one amino acid sequence Leu-Tyr-Glu-Glu-Glu-Ile-Arg-Val-Leu, wherein the amino groups may be present in the free or protonated form, and the carboxy groups may be present in the free or deprotonated form.

In summary, in particular cosmetic agents according to the invention are preferred which include at least one proteolipid of formula (I), in which R″ has at least one amino acid sequence Leu-Tyr-Glu-Glu-Glu-Ile-Arg-Val-Leu, wherein the amino groups may be present in the free or protonated form, and the carboxy groups may be present in the free or deprotonated form.

As previously mentioned, R″ is selected from the group comprising keratin or keratin hydrolysate when X=—C(O)O—.

In all other cases, the moiety R″ in formula (P-I) may stand for a peptide or a protein or a protein hydrolysate, wherein protein hydrolysates are preferred. Protein hydrolysates are product mixtures which are obtained by acidically, basically, or enzymatically catalyzed breakdown of proteins. According to the invention, protein hydrolysates of plant origin as well as animal origin may be used.

Examples of protein hydrolysates are elastin, collagen, keratin, silk, and milk protein hydrolysates, which may also be present in the form of salts. Such products are marketed under the trade names Dehylan® (Cognis), Promois® (Interorgana), Collapuron®(Cognis), Nutrilan® (Cognis), Gelita-Sol® (Deutsche Gelatine Fabriken Stoess & Co), Lexein® (Innlex), and Kerasol® (Croda), for example.

The use of protein hydrolysates of plant origin, for example soybean, almond, rice, pea, potato, and wheat protein hydrolysates, is preferred according to the invention. Such products are available under the trade names Gluadin® (Cognis), DiaMin® (Diamatt), Lexein® (Innlex), and Crotein® (Croda), for example.

Regardless of the selection of X in formula (P-I), the moiety R″ is selected from keratin or keratin hydrolysates. Preferred cosmetic agents according to the invention are characterized in that they include at least one proteolipid of formula (P-I) in which R″ stands for keratin or a keratin hydrolysate.

In particular, cosmetic agents according to the invention are preferred which include at least one proteolipid of formula (P-I), in which R^{III} means —CH₃, and R^{IV} stands for —(CH₂)ₓ—, where x=0, 1, 2, 3, 4, 5, 6, 7, 8.

In addition, particularly preferred cosmetic agents according to the invention are characterized in that they include at least one proteolipid of formula (I), in which X stands for —N⁺(CH₃)₂—CH₂—CH(OH)—CH₂— and R″ stands for —(CH₂)₁₇—CH₃.

Further cosmetic agents which are likewise preferred according to the invention are characterized in that they include at least one proteolipid of formula (P-I), in which X stands for —C(O)—O— and R″ stands for —(CH₂)₁₇—CH₃.

It has proven advantageous to use protein hydrolysates in addition to the proteolipids. Protein hydrolysates intensify the effect of the proteolipids, and their own effects are enhanced. The protein hydrolysates have been described in detail above as moiety R″. In summary, cosmetic agents according to the invention are preferred which, based on their weight, include 0.01 to 10% by weight, preferably 0.05 to 7% by weight, particularly preferably 0.1 to 5% by weight, more preferably 0.25 to 2.5% by weight, and in particular 0.5 to 2.0% by weight, of protein hydrolysate(s), preferably keratin hydrolysate(s).

For esthetic reasons, “clear” products are often preferred by consumers. Cosmetic agents preferred according to the invention are therefore characterized in that they are transparent or translucent.

Within the scope of the present invention, “transparent or translucent” is understood to mean a composition which has an NTU value of less than 100. The NTU value (Nephelometric Turbidity Unit) is a unit used in water treatment for turbidity measurements in liquids. It is the unit of a turbidity of a liquid which is measured with a calibrated nephelometer.

A further subject matter of the present invention is the use of silicones which include groups of formula (I)

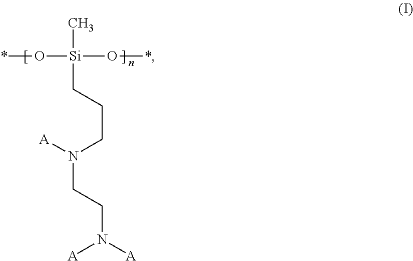

in which

n stands for values from 1 to 1000,

A stands for a group

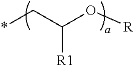

in which R1 stands for —H or —CH₃ and R stands for a straight-chain or branched alkyl moiety including 8 to 24 C atoms, and a stands for an integer from 1 to 20,
   for reducing the damage to the hair structure by external influences, in particular by UV radiation.

The use according to the invention of the silicones preferably takes place in combination with at least one copolymer having monomer units of formulas (V) and (VI)

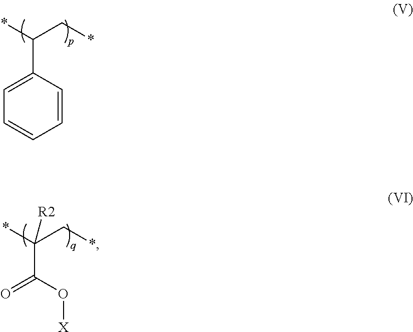

in which R2 stands for —H or —CH₃ and X stands for —H, an alkali metal cation, or a straight-chain or branched alkyl moiety including 1 to 24 C atoms.

The statements made with regard to the agents according to the invention also apply mutatis mutandis with regard to further preferred embodiments of the uses according to the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment agent which includes, in each case based on its weight,
a) 0.1 to 20% by weight of at least one silicone including groups of formula (I) 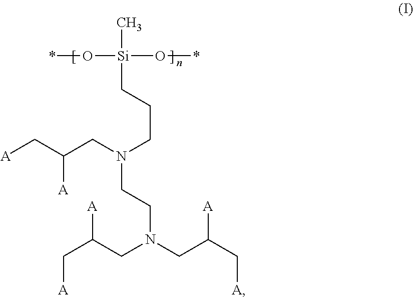
in which
n stands for values from 1 to 1000,
A stands for a group 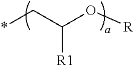
in which R1 stands for —H or —CH₃ and R stands for a straight-chain or branched alkyl moiety including 8 to 24 C atoms, and a stands for an integer from 1 to 20;
b) 0.1 to 20% by weight of at least one copolymer including monomer units of formulas (V) and (VI) 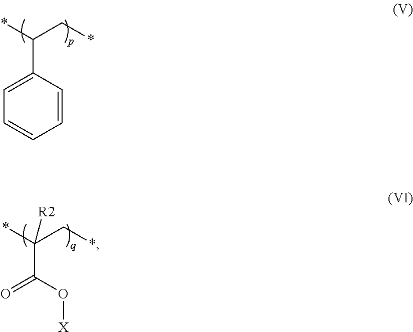
in which R2 stands for —H or —CH₃ and X stands for H, an alkali metal cation, or a straight-chain or branched alkyl moiety including 1 to 24 C atoms;
c) 0.5 to 5% by weight of 2-phenylbenzimidazole-5-sulfonic acid.

2. The hair treatment agent according to claim 1, wherein the agent includes 0.1 to 20% by weight at least one silicone including groups of formula (Ia) 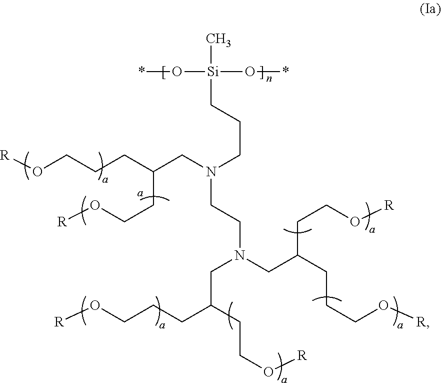
wherein R is selected from
—(CH₂)ₖ—CH₃, wherein k=7, 8, 9, 10, 11, 12, 13, 14, 15, 16, or 17, and
—CH(CH₂CH₃)—(CH₂)₅
and a stands for an integer from 1 to 20.

3. The hair treatment agent according to claim 1, wherein the agent includes 0.1 to 20% by weight at least one silicone of formula (II): 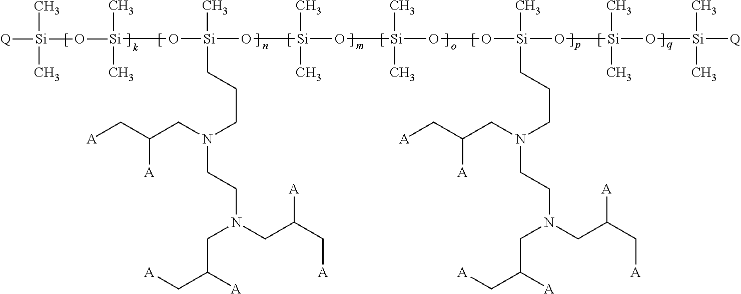
wherein
A stands for a group 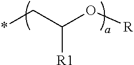
in which R1 stands for —H or —CH₃ and R stands for straight-chain or branched alkyl moiety including 8 to 24 C atoms, and a stands for an integer from 1 to 20;
Q stands for —OH, —CH₃, or —OCH₃;
the indices k, n, m, o, p, q stand for integers from 0 to 1000, with the condition that
n+p>0 and k+m+o+q>0,
wherein the units having the indices k, n, m, o, p, q may be present in the molecule as a block or statistically distributed.

4. The hair treatment agent according to claim 1, wherein the agent includes 0.1 to 20% by weight at least one silicone of formula (III): 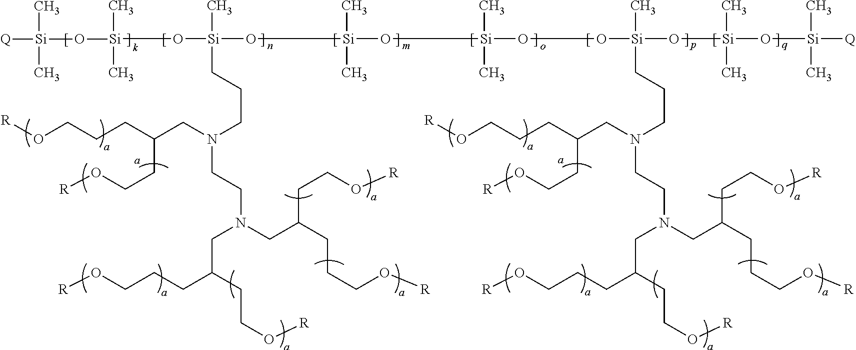
in which
R is selected from
—(CH₂)ₖ—CH₃, where k=7, 8, 9, 10, 11, 12, 13, 14, 15, 16, or 17, and
—CH(CH₂CH₃)—(CH₂)₅;
a stands for an integer from 1 to 20;
Q stands for —OH, —CH₃, or —OCH₃; and
the indices k, n, m, o, p, q stand for integers from 0 to 1000, with the condition that
n+p>0 and k+m+o+q>0,
wherein the units having the indices k, n, m, o, p, q may be present in the molecule as a block or statistically distributed.

5. The hair treatment agent according to claim 1, wherein the agent includes 0.1 to 20% by weight at least one silicone of formula (IV): 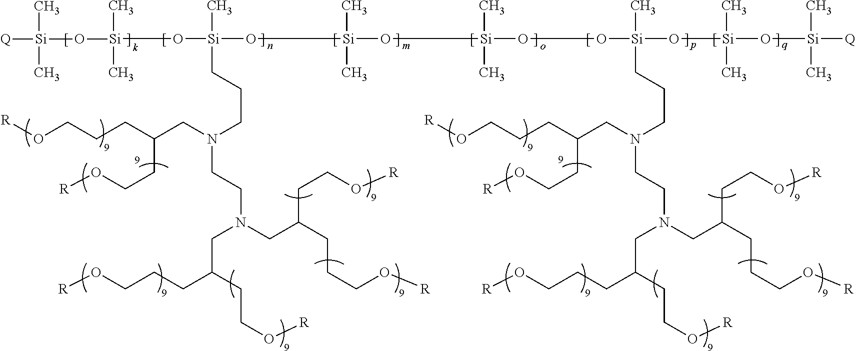
in which
R stands for —(CH₂)₁₂—CH₃ and Q stands for —OH or —OCH₃;
Q stands for —OH, —CH₃, or —OCH₃; and
the indices k, n, m, o, p, q stand for integers from 0 to 1000, with the condition that
n+p>0 and k+m+o+q>0,
wherein the units having the indices k, n, m, o, p, q may be present in the molecule as a block or statistically distributed.

6. The hair treatment agent according to claim 3, wherein the two Q's in formula (II) are selected from the following combinations:

| “Left” terminal group Q | “Right” terminal group Q |
|---|---|
| —OH | —OH |
| —CH₃ | —OH |
| —OCH₃ | —OH |
| —OH | —CH₃ |
| —CH₃ | —CH₃ |
| —OCH₃ | —CH₃ |
| —OH | —OCH₃ |
| —CH₃ | —OCH₃ |
| —OCH₃ | —OCH₃. |

7. The hair treatment agent according to claim 4, wherein the two Q's in formula (III) are selected from the following combinations:

| “Left” terminal group Q | “Right” terminal group Q |
|---|---|
| —OH | —OH |
| —CH₃ | —OH |
| —OCH₃ | —OH |
| —OH | —CH₃ |
| —CH₃ | —CH₃ |
| —OCH₃ | —CH₃ |
| —OH | —OCH₃ |
| —CH₃ | —OCH₃ |
| —OCH₃ | —OCH₃. |

8. The hair treatment agent according to claim 5, wherein the two Q's in formula (IV) are selected from the following combinations:

| “Left” terminal group Q | “Right” terminal group Q |
|---|---|
| —OH | —OH |
| —CH₃ | —OH |
| —OCH₃ | —OH |
| —OH | —CH₃ |
| —CH₃ | —CH₃ |
| —OCH₃ | —CH₃ |
| —OH | —OCH₃ |
| —CH₃ | —OCH₃ |
| —OCH₃ | —OCH₃. |

9. The hair treatment agent according to claim 1, wherein the silicone(s) is/are selected from the group consisting of silicone(s) of formulas (IIa), (IIb), (IIIa), (IIIb), (IVa), and (IVb):

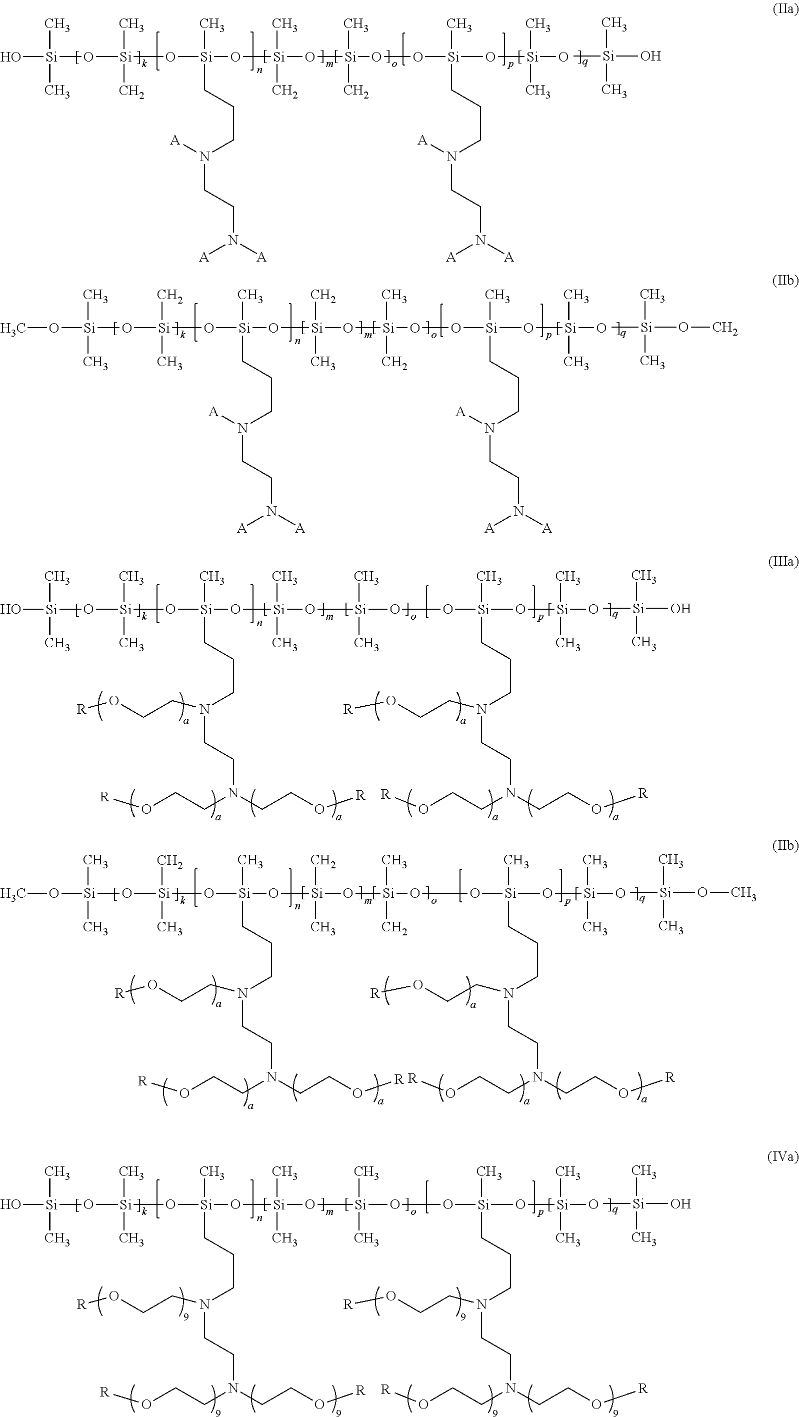

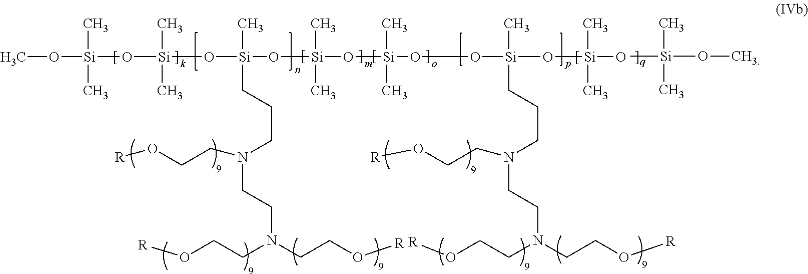

10. The hair treatment agent according to claim 1, wherein at least 50% by weight of the copolymers are composed of monomers of formulas (V) and (VI).

11. The hair treatment agent according to claim 1, wherein the copolymer(s) is/are selected from the group consisting of
copolymers of styrene and acrylic acid
copolymers of styrene and methacrylic acid
copolymers of styrene and sodium acrylate
copolymers of styrene and sodium methacrylate
copolymers of styrene and potassium acrylate
copolymers of styrene and potassium methacrylate
copolymers of styrene and acrylic acid methyl ester
copolymers of styrene and methacrylic acid methyl ester
copolymers of styrene and acrylic acid ethyl ester
copolymers of styrene and methacrylic acid ethyl ester
copolymers of styrene and acrylic acid propyl ester
copolymers of styrene and methacrylic acid propyl ester
copolymers of styrene and acrylic acid isopropyl ester
copolymers of styrene and methacrylic acid isopropyl ester
copolymers of styrene and acrylic acid (n-butyl) ester
copolymers of styrene and methacrylic acid (n-butyl) ester
copolymers of styrene and acrylic acid and methacrylic acid
copolymers of styrene and acrylic acid and sodium acrylate
copolymers of styrene and acrylic acid and sodium methacrylate
copolymers of styrene and acrylic acid and acrylic acid methyl ester
copolymers of styrene and acrylic acid and methacrylic acid methyl ester
copolymers of styrene and acrylic acid and acrylic acid ethyl ester
copolymers of styrene and acrylic acid and methacrylic acid ethyl ester
copolymers of styrene and acrylic acid and acrylic acid (n-butyl) ester
copolymers of styrene and acrylic acid and methacrylic acid (n-butyl) ester
copolymers of styrene and methacrylic acid and sodium acrylate
copolymers of styrene and methacrylic acid and sodium methacrylate
copolymers of styrene and methacrylic acid and acrylic acid methyl ester
copolymers of styrene and methacrylic acid and methacrylic acid methyl ester
copolymers of styrene and methacrylic acid and acrylic acid ethyl ester
copolymers of styrene and methacrylic acid and methacrylic acid ethyl ester
copolymers of styrene and methacrylic acid and acrylic acid (n-butyl) ester
copolymers of styrene and methacrylic acid and methacrylic acid (n-butyl) ester
copolymers of styrene and acrylic acid and methacrylic acid and sodium acrylate
copolymers of styrene and acrylic acid and methacrylic acid and sodium methacrylate
copolymers of styrene and acrylic acid and methacrylic acid and acrylic acid methyl ester
copolymers of styrene and acrylic acid and methacrylic acid and methacrylic acid methyl ester
copolymers of styrene and acrylic acid and methacrylic acid and acrylic acid ethyl ester
copolymers of styrene and acrylic acid and methacrylic acid and methacrylic acid ethyl ester
copolymers of styrene and acrylic acid and methacrylic acid and acrylic acid (n-butyl) ester, and
copolymers of styrene and acrylic acid and methacrylic acid and methacrylic acid (n-butyl) ester.

12. The hair treatment agent according to claim 1, wherein the agent includes 0.5 to 17.5% by weight at least one copolymer having monomer units of formulas (V) and (VIa) 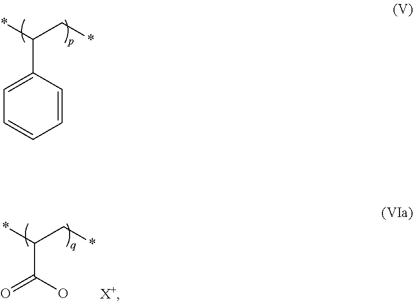
wherein X stands for H⁺ or Na⁺.

13. The hair treatment agent according to claim 1, wherein the agent includes 0.55 to 4.5% by weight 2-phenylbenzimidazole-5-sulfonic acid.

14. The hair treatment agent according to claim 1, wherein the weight ratio of ingredients b) to a) is in the range of 1:50 to 50:1.

15. The hair treatment agent according to claim 1, wherein the weight ratio of ingredients a) to c) is in the range of 1:50 to 50:1.

16. A method for reducing the damage to the hair structure by external influences, in particular by UV radiation, including:
applying to the hair silicones which include groups of formula (I) 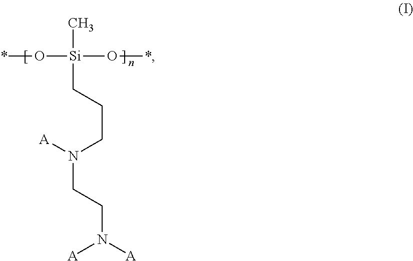
in which
n stands for values from 1 to 1000,
A stands for a group 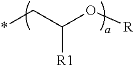
in which R1 stands for —H or —CH₃ and R stands for a straight-chain or branched alkyl moiety including 8 to 24 C atoms, and a stands for an integer from 1 to 20.
